(19) United States
(12) Patent Application Publication
(10) Pub. No.: US 2025/0109381 A1
(43) Pub. Date: Apr. 3, 2025

(54) COMPOSITION FOR CULTRING NK CELLS, COMPRISING ALLOFERON, AND METHOD FOR CULTURING NK CELLS USING SAME

(71) Applicant: NK MEDICS INC., Seoul (KR)

(72) Inventors: Gwang Jin YOO, Seongnam-si (KR); Woon Young SEO, Seoul (KR)

(21) Appl. No.: 18/727,799

(22) PCT Filed: Jan. 28, 2022
(86) PCT No.: PCT/KR2022/001574

§ 371 Date: Jul. 10, 2024

(30) Foreign Application Priority Data
Jan. 28, 2022 (KR) 10-2022-0012882

(51) Int. Cl. C12N 5/0783

(52) U.S. Cl. CPC C12N 5/0646; C12N 2501/2302; C12N 2501/2312; C12N 2501/2315; C12N 2501/33; C12N 2501/515; C12N 2501/998

(57) ABSTRACT

The present invention relates to a composition for culturing NK cells, comprising alloferon, which increases the yield of NK cells compared to when using a conventional NK cell medium, due to the addition made to the medium during the culture of NK cells, and can lead to the activation of NK cells.

### TECHNICAL FIELD

The present invention relates to a composition for culturing NK cells, comprising alloferon, which increases the yield of NK cells compared to when using a conventional NK cell medium, due to the addition made to the medium during the culture of NK cells, and can lead to the activation of NK cells.

### BACKGROUND ART

Several natural components containing substances from animal and plant tissues, including insects, that can stimulate the effectiveness of the immune system, are known. Among them, alloferon is a linear peptide having a molecular weight of 1265 daltons derived from the immune material of infected flies. According to various studies so far, alloferon has been found to exhibit a wide range of antiviral, anticancer, anti-inflammatory and anti-allergic effects through immune modulating effects. Alloferon is rapidly absorbed into the body and remains stably for a long period of time, thereby maximizing its efficacy. In particular, alloferon is marketed as a Class I new drug in the classification of non-toxic drugs.

Meanwhile, NK cells are one of the immune cells included in lymphocytes, and are called ‘natural killer (NK) cells’ due to the characteristic that they have the ability to kill external enemies from the moment they are produced. NK cells act widely within the body, and when abnormal cells such as cancer cells and virus-infected cells are discovered, they can first attack them independently. This independence and immediate effect are the biggest characteristics of NK cells. Since NK cells do not have an antigen-antibody response, they can directly attack abnormal cells freely and flexibly, and are attracting attention as a cell therapy agent because the capability is remarkable even among immune cells that attack cancer cells.

Korean Patent Publication No. 10-0394864 discloses the use of alloferon as an anticancer agent. However, it does not disclose that its use as a culture material for NK cells can have an effect on increasing the growth rate of NK cells or on NK cell activity.

### DETAILED DESCRIPTION OF THE INVENTION

### Technical Problem

One aspect provides a composition for culturing NK cells, comprising a polypeptide represented by an amino acid sequence of any one of SEQ ID NOs: 1˜4 or a combination thereof.

In one embodiment, the composition for culturing NK cells includes a polypeptide represented by an amino acid sequence of any one of SEQ ID NOs: 1˜4 or a combination thereof; IL-2; and IL-12, IL-15, OKT-3, or a combination thereof.

In another embodiment, the composition for culturing NK cells includes FBS or autologous plasma, human serum albumin, insulin, transferrin or a combination thereof.

In another embodiment, the composition for culturing NK cells includes a polypeptide represented by an amino acid sequence of any one of SEQ ID NOs: 1˜4 or a combination thereof; IL-2, IL-12, IL-15, and OKT-3.

In another embodiment, the composition for culturing NK cells contains 6 mg/L to 40 mg/L of the polypeptide.

In another embodiment, the composition for culturing NK cells include IL-2 in an amount of 0.5 mg/L to 5 mg/L relative to the total volume of the cell culture medium; IL-12 in an amount of 0.5 mg/L to 5 mg/L relative to the total volume of the cell culture medium; IL-15 in an amount of 5 mg/L to 15 mg/L relative to the total volume of the cell culture medium; and OKT-3 in an amount of 10 mg/L to 30 mg/L relative to the total volume of the cell culture medium.

In another embodiment, the composition for culturing NK cells includes the FBS or autologous plasma in an amount of 1 v/v % to 20 v/v % relative to the total volume of the culture medium; the human serum albumin in an amount of 100 mg/L to 3,000 mg/L relative to the total volume of the culture medium; the insulin in an amount of 5 mg/L to 15 mg/L relative to the total volume of the culture medium; and the transferrin in an amount of 5 mg/L to 15 mg/L relative to the total volume of the culture medium. Another aspect provides a method of culturing NK cells in a medium containing the composition for culturing NK cells.

### Technical Solution

According to one aspect, there is provided a composition for culturing NK cells, comprising a polypeptide represented by an amino acid sequence of any one of SEQ ID NOs: 1˜4 or a combination thereof; IL-2, IL-12, IL-15, OKT-3, or a combination thereof.

In the present specification, each polypeptide is an alloferon, and specifically, the polypeptide represented by SEQ ID NO: 1 may be named alloferon 1, the polypeptide represented by SEQ ID NO: 2 may be named alloferon 2, the polypeptide represented by SEQ ID NO: 3 may be named alloferon 3, and the polypeptide represented by SEQ ID NO: 4, may be named alloferon 4.

The polypeptide may be a combination of one or more selected from the group consisting of alloferon 1, alloferon 2, alloferon 3, and alloferon 4. Specifically, in the composition for culturing NK cells according to one embodiment, the polypeptide may include alloferon 1, alloferon 2, alloferon 3, and alloferon 4 as individual substances, and may include a combination of alloferons 1 and 2, a combination of alloferons 1 and 3, and a combination of alloferons 1 and 4, a combination of alloferons 2 and 3, a combination of alloferons 2 and 4, a combination of alloferons 3 and 4, or a combination of alloferons 1, 2, 3, and 4.

The composition for culturing NK cells may further include one or more components selected from the group consisting of IL-2 (interleukin-2), IL-12(interleukin-12), IL-15 (interleukin-15), and OKT-3 (Anti-CD3 antibody). Specifically, the composition for culturing NK cells may, in addition to IL-2, further include one or more components selected from the group consisting of IL-12, IL-15, and OKT-3. Here, IL-2 may be included in an amount of 0.5 mg/L to 5 mg/L, 0.5 mg/L to 4 mg/L, 1 mg/L to 3 mg/L, 1 mg/L to 2.5 mg/L, 1.5 mg/L to 2.5 mg/L, or 2 mg/L relative to the total volume of the culture medium. IL-12 may be included in an amount of 0.5 mg/L to 5 mg/L, 0.5 mg/L to 4 mg/L, 1 mg/L to 3 mg/L, 1 mg/L to 2.5 mg/L, 1.5 mg/L to 2.5 mg/L, or 2 mg/L relative to the total volume of the culture medium. IL-15 may be included in an amount of 5 mg/L to 15 mg/L, 7 mg/L to 12 mg/L, 8 mg/L to 11 mg/L, or 10 mg/L relative to the total volume of the culture medium. OKT-3 may be included in an amount of 10 mg/L to 30 g/L, 12 mg/L to 28 mg/L, 15 mg/L to 25 mg/L, 18 mg/L to 22 mg/L, 19 mg/L to 21 mg/L, or 20 mg/L relative to the total volume of the culture medium. The composition for culturing NK cells may include IL-2, but may not include IL-12, IL-15, and OKT-3. If the composition includes the combination of IL-2 and alloferon, it can significantly increase the culture efficiency of NK cells, and therefore, it may be advantageous in reducing costs and minimizing side effects due to the inclusion of unnecessary components. However, in order to solve problems such as culture stability, IL-12, IL-15, or OKT-3 can be added to IL-2 and alloferon, or the amount can be appropriately increased or decreased.

The composition for culturing NK cells may further include any one or more components selected from the group consisting of FBS or autologous plasma, human serum albumin, insulin, and transferrin. The FBS or autologous plasma may be included in an amount of 1 v/v % to 20 v/v %, 5 v/v % to 15 v/v %, 8 v/v % to 12 v/v %, or 10 v/v % relative to the total volume of the culture medium. The human serum albumin may be included in an amount of 100 mg/L to 3,000 mg/L, 500 mg/L to 2,500 mg/L, 1,000 mg/L to 2,500 mg/L, 1,500 mg/L to 2,500 mg/L, or 1,800 mg/L to 2,200 mg/L, or 2,000 mg/L relative to the total volume of the culture medium. The insulin may be included in an amount of 5 mg/L to 15 mg/L, 7 mg/L to 12 mg/L, 8 mg/L to 11 mg/L, or 10 mg/L relative to the total volume of the culture medium. The transferrin may be included in an amount of 5 mg/L to 15 mg/L, 7 mg/L to 12 mg/L, 8 mg/L to 11 mg/L, or 10 mg/L relative to the total volume of the culture medium.

The composition for culturing NK cells includes a medium commonly known as a basic medium, for example, RPMI 1640 medium, but is not limited thereto.

The composition for culturing NK cells includes the polypeptide in an amount of 6 mg/L to 40 mg/L, 8 mg/L to 30 mg/L, 8 mg/L to 25 mg/L, 8 mg/L to 20 mg/L, 8 mg/L to 15 mg/L, 8 mg/L to 12 mg/L, or 10 mg/L.

The content of each component of the composition for culturing NK cells may be an amount suitable for culturing NK cells at a density of 1×10⁴ cells/ml to 1×10⁶ cells/ml, or 1×10⁵ cells/ml.

According to the other aspect, there is provided a method of culturing NK cells, comprising the steps of: obtaining NK cells from blood isolated from an individual; and culturing NK cells in a medium for culturing NK cells as set forth in claim 1.

When NK cells are cultured using the composition for NK cell culture of the present invention, the yield of NK cells can be increased by 3-fold or more compared to the case where alloferon is not treated. In addition, the proliferation rate of NKT cells can be reduced to 0.6-fold or less, 0.55-fold or less, 0.5-fold or less, 0.45-fold or less, or 0.4-fold or less compared to the case where alloferon is not treated, which is advantageous for NK cell culture. (***removed after confirming the function of NKT cells)

The composition for culturing NK cells of the present invention can also activate NK cells. Specifically, when cultured in a medium containing the composition for culturing NK cells of the present invention, compared to before culture, the expression level of NKp30 may be 45% or more, preferably 55% or more, the expression level of NKp44 may be 50% or more, preferably 60% or more, the expression level of NKp46may be 65% or more, preferably 80% or more, and the expression level of perforin may be 90% or more, preferably 99% or more. In addition, when cultured in a medium containing the composition for culturing NK cells of the present invention, the amount of IFN-gamma secreted may be increased by 3 times or more, 4 times or more, 5 times or more, or 6 times compared to when cultured in a medium not containing alloferon.

The composition for culturing NK cells of the present invention can be used as a pharmaceutical composition for preventing or treating cancer comprising natural killer cells as an active ingredient.

The pharmaceutical composition means a “cellular therapeutic agent”. As used herein, the term “cellular therapeutic agent” refers to cells and tissues prepared by isolation from an individual, culture and special operations, and means a pharmaceutical product (US FDA regulations) which is used for the purposes of treatment, diagnosis and prevention and which is obtained through a series of actions, including growing and screening living autologous or allogenic cells in vitro in order to restore the structure and function of the cells or changing the biological characteristics of cells by any other methods.

As used herein, the term “prevention” refers to any action that inhibits or delays the onset of cancer diseases by administration of the pharmaceutical composition.

Further, as used herein, the term “treatment” refers to any action that ameliorates or beneficially change symptoms of cancer diseases by administration of the pharmaceutical composition.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, excipients or diluents suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition of the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Parenteral administration is preferred.

As used herein, the term “parenteral” includes subcutaneous, intradermal, intravenous, intramuscular, intraarticular, intrabursal, intrasternal, intradural, intralesional, and intracranial injection or infusion techniques.

The pharmaceutical composition of the present invention may vary depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way.

### Advantageous Effects

By culturing NK cells in a medium containing a composition for culturing NK cells according to one aspect, the yield of NK cells can be increased.

In addition, NK cells cultured in the presence of the composition for culturing NK cells according to one aspect have an excellent cancer killing effect, which is beneficial for cultivating excellent NK cells.

### BRIEF DESCRIPTION OF THE DRAWINGS

FIG. 1 shows an image of the shape of NK cells cultured in NK cell culture medium for 14 days.

FIG. 2 is a graph showing the proliferation fold of cells according to the conditions of the NK cell culture medium based on the number of days of culture.

FIG. 3 is a graph showing the cell count proliferation rate according to alloferon peptide treatment concentration.

FIG. 4 graphically shows the NK cell proliferation rate according to NK cell culture conditions.

FIG. 5 is a FACS analysis result showing the increase rate of NK cells from the beginning of culture (day 0) to the end of culture (day 21) when treated with alloferon 1.

FIG. 6 is an image confirming the level of NK cell activity by FACS when cultured in NK cell culture medium according to one embodiment.

FIG. 7 graphically shows the results of confirming the cytotoxicity of NK cells against cancer cells according to each NK cell culture medium condition.

FIG. 8 is the result of FACS analysis showing the results of cell granulation analysis according to each NK cell culture medium condition.

### DETAILED DESCRIPTION OF THE EMBODIMENTS

Hereinafter, the present invention will be described in more detail with reference to Experimental Examples. The following Experimental Examples are only for illustrating the present invention in more detail, and according to the gist of the present invention, the scope of the present invention is not limited by the Experimental Examples.

### Example 1. Obtainment of NK Cells and Preparation of NK Cell Culture Medium

### 1-1. Obtainment of NK Cells

30 ml of blood was collected from a human blood donor, RosetteSep NK enrichment antibody cocktail (50 ul/ml) was added thereto, and then diluted 1:1 v/v with phosphate buffer solution (PBS). The blood was centrifuged at 1600 rpm for 20 minutes using a density gradient centrifugation (Ficoll-Plaque) to separate the cell layer containing NK cells. The process of washing and centrifuging this with PBS was repeated twice. Then, the number of cells was counted by staining with trypan blue, and the average number of cells obtained was 1.6˜3.0×10⁵ cell/ml, and the cells were cultured in NK cell culture medium.

### 1-2. Preparation of NK Cell Culture Medium

The NK cell culture medium used was a combination of various substances that were conventionally known to be necessary for NK culture. Specifically, RPMI1640 was used as the basic medium, to which 10% (v/v) FBS (F2442, Merck) or autologous plasma, human serum albumin 2,000 mg/L, recombinant human insulin 10 mg/L, recombinant human transferrin 10 mg/L, recombinant human interleukin 2 (IL-2) 2 mg/L, recombinant human interleukin 12 (IL-12) 2 mg/L, recombinant human interleukin 15 (IL-15) 10 mg/L and anti-CD3 antibody (OKT-3) 20 mg/L were mixed. This was used as the NK cell culture medium of Example 1, to which alloferon was further mixed and used at 0.1 to 100 mg/L as needed.

### 1-3. Confirmation of NK Cell Culture Effect in NK Cell Culture Medium

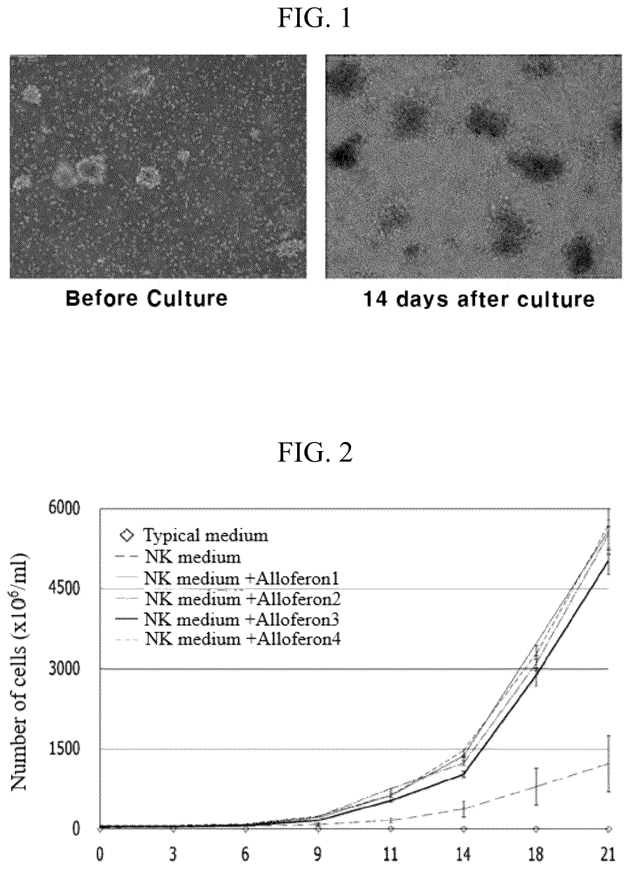

NK cells were cultured in the NK cell culture medium prepared according to Example 1. As a result, as shown in FIG. 1, it was confirmed that a very small number of NK cell-containing clusters exist in whole blood cells before culture, but after culture, the size of these clusters increases and the number increases.

### Test Example 1. Confirmation of NK Cell Culture Effect Due to the Addition of Alloferon

The cell proliferation effect due to the addition of alloferon to each medium prepared in Example 1 was confirmed by Cell proliferation assay (incorporation of [³H]-thymidine).

Specifically, NK cells (1×10⁵ cells/ml) were placed in 96-well plates, and then treated with 10 mg/L each of alloferons 1 to 4 in the NK cell culture medium of Example 1, and then cultured in an environment at 5% CO₂, 37° C. for 48 hours or 21 days. After the culture, [³H]-thymidine (37 mBq) was added at 0.2 μCi per well and cultured for 8 hours. Both cells and medium were transferred to a glass fiber filter (size 90.120 mm; Wallac, Turkr, Finland) using a 96-well harvester. After drying the filter, a scintillator sheet (Meltilex, Wallac) was melted to coat the filter. After that, the radioactivity was measured using 1450 MicroBeta TriLux (PerkinElmer Life and Analytical Sciences). The amino acid sequences of alloferons 1 to 4 added thereto are shown in Table 1 below.

TABLE 1

| | |
|---|---|
| Alloferon 1 (SEQ ID NO: 1) | HGVSGHGQHGVHG |
| Alloferon 2 (SEQ ID NO: 2) | GVSGHGQHGVHG |
| Alloferon 3 (SEQ ID NO: 3) | SGHGQHGV |
| Alloferon 4 (SEQ ID NO: 4) | VSGHGQHGVH |

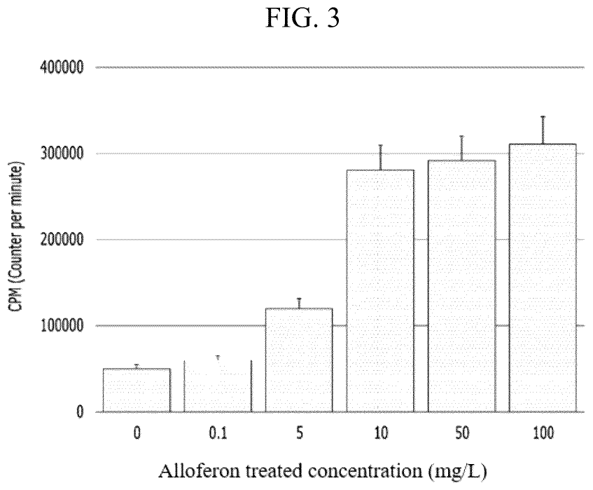

As can be seen in FIG. 2, when the NK cell culture medium was treated with alloferons 1 to 4 at 10 mg/L each and cultured for 21 days, the number of NK cells increased by an average of about 1430 times. On the other hand, it was confirmed that in a typical medium, which is a control group, NK cells hardly grew, and even when cultured in the NK culture medium of Example 1 containing no alloferon, NK cells grew, but grew to approximately ⅕ only for 21 days. Similarly, the cells were treated with different concentrations of alloferon 1 and cultured for 21 days. As a result, as can be seen in FIG. 3, it was confirmed that as the amount of alloferon treated increased from 0.1 to 100 mg/L, the amount of NK cells increased, but the maximum value was reached at a concentration of 10 mg/L.

### Test Example 2. Confirmation of Changes During Co-Treatment of Alloferon and Other Interleukins

In order to identify factors that imparts the influence other than alloferon during NK cell culture, the NK cell proliferation effect was confirmed after co-treatment with various combinations of interleukins. Except for the medium composition, the NK cell culture method was the same as in Test Example 1, and the cells were cultured for 21 days (*** random period).

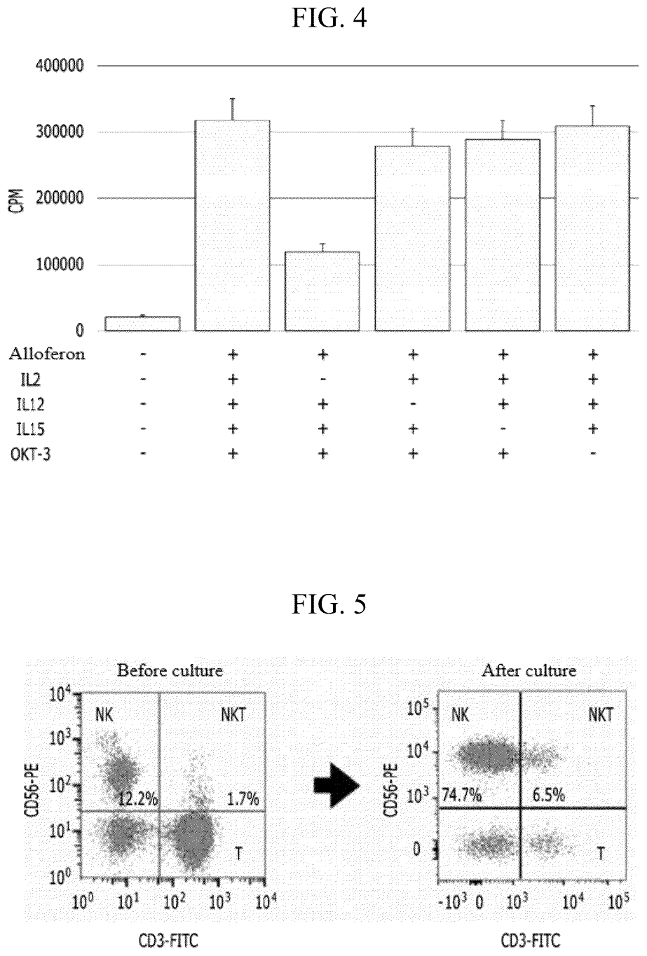

As a result, as can be seen in FIG. 4, it was confirmed that when only alloferon 1 was added at 10 mg/L, NK cell proliferation was induced. However, it was confirmed that if IL-2 was excluded, the cell proliferation effect was significantly reduced.

### Test Example 3. Confirmation of NK Cell Increase Rate

Flow Cytometry (FACS analysis) analysis was performed to confirm the NK cell increase rate. Specifically, NK cells were cultured for 21 days in a medium containing 10 mg/L each of Test Example 1, Example 1 (untreated), and Alloferon 1 to 4 in Example 1. Cells before and after culture were collected by centrifugation at 1,400 rpm for 5minutes, and the collected cells were washed twice using PBS. Then, the cells were suspended in 1 ml of PBS, and the number of living cells was measured through Trypan blue staining. The cells were dispensed into FACS tubes at 1×10⁶ cells/tube, and 2 ml of PBS was added. Then, centrifugation was performed at 400 g and 4° C. for 5 minutes, and the supernatant excluding cells was removed. 100 μl of 1% albumin was added to the remaining cell pellet, and antibodies were added and mixed with PBS. NK cells were treated with antibodies (CD3-FITC, CD56-PE), reacted in a light shielding state for 30 minutes, and then the cells were washed twice with PBS. NK cells were suspended in an appropriate amount of PBS, and then the data were compared and analyzed using FACSVerse (BD Biosciences, San Diego, CA, USA). The results are shown in Table 2, Table 3, and FIG. 5.

TABLE 2

| Alloferon 1 | Before culture | After culture |
|---|---|---|
| NK | 12.2% | 74.7% |
| NKT | 1.7% | 6.5% |

TABLE 3

| Kind | Alloferon 1 | Alloferon 2 | Alloferon 3 | Alloferon 4 | Untreated |
|---|---|---|---|---|---|
| NK | 74.7% | 73.8% | 69.7% | 71.5% | 21.4% |
| NKT | 6.5% | 12.4% | 13.2% | 8.4% | 25.7% |

10 mg/L of Alloferon 1 was added to the NK cell culture medium of Example 1,and the increase rate of NK cells and other immune cells was confirmed from the start of culture (day 0) to the end of culture (day 21). As a result, it was confirmed that the number of NK cells increased 1,430 times compared to when grown in a typical medium (RPMI), and among them, the NKT cells increased 560-fold and the NK cells increased 8,790-fold, so the increase in NK cells was significantly higher compared to the control group after culture.

Similarly, the NK cell proliferation rate was confirmed after treatment with each of alloferons 2 to 4, and is shown in Table 3 below. It can be confirmed that, compared to the group cultured in the NK cell culture medium of Example 1 without alloferon treatment, the proliferation rate of NK cells increased by more than three times when treated with alloferon.

### Test Example 4. Comparison of NK Cell Activation Receptor Expression

Based on the results of Test Example 3, receptor expression was confirmed and compared to measure the activity of NK cells. Specifically, as in Test Example 3, NK cells were cultured in each medium, and in order to measure the activity of NK cells proliferated for 21 days, the proliferated NK cells were isolated by flow cytometry. The expression of activating markers for these cells was further confirmed. The cultured NK cells were washed twice using PBS, then dispensed into a FACS tube at 1×10⁶ cells/tube, and 2 ml of PBS was added. Then, centrifugation was performed at 400 g and 4° C. for 5 minutes, and the supernatant excluding cells was removed. 100 μl of 1% albumin was added to the remaining cell pellet, sorted using antibodies (CD3-FITC, CD56-PE) together with PBS, and then isolated according to surface markers. In addition to the isolated cells, NK cells were treated with antibodies (Anti-NKp30, anti-NKp44, anti NKp46, and Perforin) to confirm the expression of activating receptors (NKp30, NKp44, NKp46) and Perforin on the surface of NK cells, and reacted in a light shielding state for 30 minutes, and the cells were washed twice again using PBS. NK cells were suspended in an appropriate amount of PBS, and the data were compared and analyzed using FACS AriaII (BD Biosciences, San Diego, CA, USA).

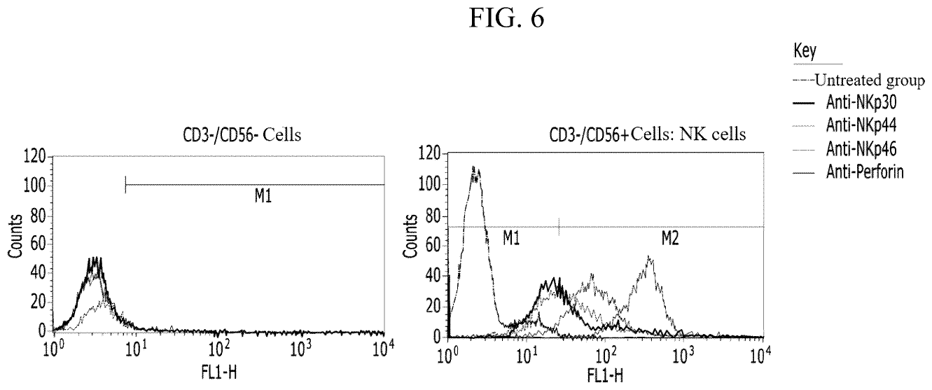

As a result, as shown in FIG. 6, Cells cultured in NK medium to which alloferon 1 was added showed higher NK activity than the typical medium to which alloferon was not added or the NK cell culture medium of Example 1.

Similarly, the same test was performed for alloferons 2 to 4, and as a result, as can be seen in Table 4 below, it showed higher NK activity that those in the typical medium to which alloferon was not added or the NK cell culture medium of Example 1.

TABLE 4

| Kind | NKp30 | NKp44 | NKp46 | Perforin |
|---|---|---|---|---|
| Typical | 0% | 0% | 0.3% | 1.4% |
| culture | | | | |
| NK culture | 22.3% | 18.9% | 31.3% | 33.5% |
| Alloferon 1 | 57.4% | 60.3% | 88.4% | 99.8% |
| Alloferon 2 | 53.2% | 50.9% | 78.6% | 99.9% |
| Alloferon 3 | 47.8% | 51.3% | 69.1% | 90.4% |
| Alloferon 4 | 61.1% | 68.2% | 82.5% | 99.8% |

### Test Example 5. Confirmation of the Secretion Rate of IFN-Gamma of NK Cells

IFN-r is an indicator of the activity of NK cells, and it is considered as the secretion amount is higher, the cytotoxicity is higher and the differentiation ability increases.

To confirm this, the isolated NK cells were washed and then treated with 10 mg/L of IL-2 in a typical medium, further treated with alloferons 1 to 4 (0.1 to 100 ng/ml, or 100 mg/L) depending on the test group, and placed in a 24-well plate (1×10⁵ cells/well) and reacted in an environment of 5% CO₂ and 37° C. for 48 hours. IFN-y secreted from NK cells was measured in the culture medium in which the cells were cultured, using a Human IFN-y ELISA kit (BD Biosciences, San Diego, CA, USA). 100 μl of capture antibody diluted in coating buffer was added to each well of a 96-well plate for ELISA, then sealed and left overnight at 4° C. It was washed three times using wash buffer, and all remaining buffer was removed. 200 μl of assay diluent was added to each well and reacted at room temperature for 1 hour for blocking. It was washed again three times using wash buffer, and all remaining buffer was removed. 100 μl of each prepared IFN-y standard and culture supernatants were added, the plate was sealed, and the reaction was performed at room temperature for 2 hours. It was washed 5 times using wash buffer, and all remaining buffer was removed. 100 μl of Working detector (Detection Antibody+SAv-HRP reagent) was added to each well, the plate was sealed, and reaction was performed at room temperature for 1 hour. It was washed 7 times using wash buffer, and all remaining buffer was removed. 100 μl of Substrate solution was added to each well and the reaction was performed at room temperature while shielding light for 30 minutes. 50 μl of Stop solution was added to each well, and the absorbance was measured at 450 nm within 30 minutes.

The results are shown in Tables 5 and 6 below.

TABLE 5

| | | | | | | |
|---|---|---|---|---|---|---|
| Alloferon 1 treated | 0 | 0.1 | 5 | 10 | 50 | 100 |
| concentration (mg/L) | | | | | | |
| IFN-r(ug/L) | 0.03 | 0.13 | 1.82 | 2.33 | 4.86 | 6.32 |
| S.D | 0.01 | 0.03 | 0.04 | 0.07 | 0.12 | 0.33 |

TABLE 6

| Kind of | | | | | |
|---|---|---|---|---|---|
| alloferon | Alloferon | Alloferon | Alloferon | Alloferon | |
| (100 mg/L) | 1 | 2 | 3 | 4 | IL-2 |
| IFN- | 6.32 | 4.87 | 3.31 | 5.84 | 1.02 |
| r(ug/L) | | | | | |
| S.D | 0.33 | 0.42 | 0.23 | 0.29 | 0.54 |

### Test Example 6. Confirmation of Cytotoxicity of NK Cells to Cancer Cells

To evaluate the cytotoxic ability against cancer cells, K562 (Korea Cell Line Bank), which is a blood cancer cell line, was furnished and used. The blood cancer cell line K562 used as a target cell was counted with a hemocytometer, and 1.0×10⁴ cells were used in a ratio of 1. In the NK cells that had completed differentiation and were used as effector cells were counted in the NK medium containing alloferon 1, Cells were counted from cells cultured by adding alloferon 1 to NK medium (NK medium+alloferon). cells cultured only in the NK medium of Example 1 (NK medium). And NK cells (control group) isolated from human blood, and cell counts of 1×10⁴, 5×10⁴, and 10×10⁴ were used as ratios. Target cells and effector cells were distributed in a 96-well plate with RPMI1640 medium (serum free) at a ratio of 1:1, 5:1, 10:1 (E: T ratio), and co-cultured in an incubator at 37° C. for 4 hours. After that, the percentage of dead cells was measured using the MTT assay (DoGen, EZ-cytox) Kit with a 450 nm filter in the Bio-Rad Microplate Reader and i-Mark to confirm cell-mediated cytotoxicity. Spontaneous release used was those containing only media, and maximum release used was those containing 2% Triton x-100.

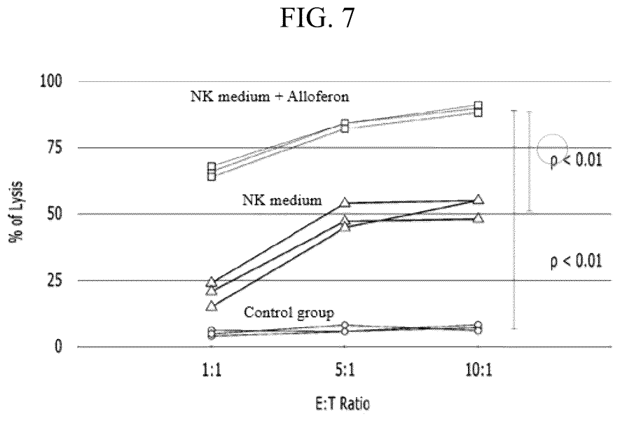

The results are shown in FIG. 7.

### Test Example 7. Analysis of Cell Granulation

To measure granulation of NK cells, the expression of CD107a lysosome-associated membrane protein-1 (LAMP-1) was measured using flow cytometry. Target cells were dispensed into 96-well round bottom plates at 1×10⁵ cells each, and the NK cells of the NK medium+alloferon of Example 1, the NK medium of Example 1, and the control group were distributed into the wells to match an E:T ratio (1:1). Anti-CD107a-PE (BD Biosciences, San Diego, CA, USA) antibody was added and then cultured in an environment of 5% CO₂ and 37° C. for 4 hours. After washing twice with PBS, the cells were treated with anti-CD56-APC (BD Biosciences, San Diego, CA, USA) antibody, reacted while blocking light for 30 minutes, and then again washed twice incubated with PBS. NK cells were suspended in an appropriate amount of PBS, then measured using

FACSVerse (BD Biosciences, San Diego, CA, USA), and the data were compared and analyzed.

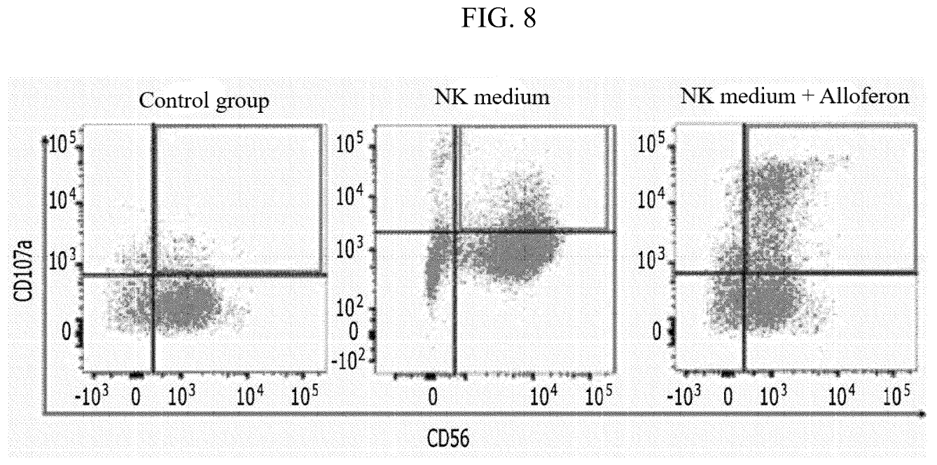

As a result, as shown in FIG. 8, the expression of CD107a, which increases when NK cells secrete granules such as perforin and granzymes, was measured to hereby measure the cytotoxicity of NK cells.

### INDUSTRIAL APPLICABILITY

The present invention has industrial applicability in the NK cell culture industry.

Sequence List Free Text

| | |
|---|---|
| <110> | NK Medics |
| <120> | Composition for culturing NK cells containing alloferon and |
| | method for culturing NK cells using the same |
| <130> | PP220003 |
| <160> | 4 |
| <170> | KoPatentIn 3.0 |
| <210> | 1 |
| <211> | 13 |
| <212> | PRT |
| <213> | Artificial Sequence |
| <220> | |
| <223> | engineered from known peptides |
| <400> | 1 |

His Gly Val Ser Gly His Gly Gln His Gly Val His Gly
1 5 10

| | |
|---|---|
| <210> | 2 |
| <211> | 12 |
| <212> | PRT |
| <213> | Artificial Sequence |
| <220> | |
| <223> | engineered from known peptides |
| <400> | 2 |

Gly Val Ser Gly His Gly Gln His Gly Val His Gly
1 5 10

| | |
|---|---|
| <210> | 3 |
| <211> | 8 |
| <212> | PRT |
| <213> | Artificial Sequence |
| <220> | |
| <223> | engineered from known peptides |
| <400> | 3 |

Ser Gly His Gly Gln His Gly Val
1 5

| | |
|---|---|
| <210> | 4 |
| <211> | 10 |
| <212> | PRT |
| <213> | Artificial Sequence |
| <220> | |
| <223> | engineered from known peptides |
| <400> | 4 |

Val Ser Gly His Gly Gln His Gly Val His
1 5 10

1. A composition for culturing NK cells, comprising a polypeptide represented by an amino acid sequence of any one of SEQ ID NOs: 1˜4 or a combination thereof.

2. The composition for culturing NK cells according to claim 1, further comprising IL-2, IL-12, IL-15, OKT-3, or a combination thereof.

3. The composition for culturing NK cells according to claim 1, further comprising a polypeptide represented by an amino acid sequence of any one of SEQ ID NOs: 1˜4 or a combination thereof; IL-2; and IL-12, IL-15, OKT-3, or a combination thereof.

4. The composition for culturing NK cells according to claim 1, further comprising FBS or autologous plasma, human serum albumin, insulin, transferrin or a combination thereof.

5. The composition for culturing NK cells according to claim 1, comprising a polypeptide represented by an amino acid sequence of any one of SEQ ID NOs: 1˜4 or a combination thereof; IL-2, IL-12, IL-15, and OKT-3.

6. The composition for culturing NK cells according to claim 1, wherein the polypeptide or a combination thereof is included in an amount of 6 mg/L to 40 mg/L.

7. The composition for culturing NK cells according to claim 1, wherein the IL-2 is included in an amount of 0.5 mg/L to 5 mg/L relative to the total volume of the cell culture medium, the IL-12 is included in an amount of 0.5 mg/L to 5 mg/L relative to the total volume of the cell culture medium, the IL-15 is included in an amount of 5 mg/L to 15 mg/L relative to the total volume of the cell culture medium, and the OKT-3 is included in an amount of 10 mg/L to 30 mg/L relative to the total volume of the cell culture medium.

8. The composition for culturing NK cells according to claim 3, wherein the FBS or autologous plasma is included in an amount of 1 v/v % to 20 v/v % relative to the total volume of the culture medium, the human serum albumin in an amount of 100 mg/L to 3,000 mg/L relative to the total volume of the culture medium, the insulin in an amount of 5 mg/L to 15 mg/L relative to the total volume of the culture medium, and the transferrin in an amount of 5 mg/L to 15 mg/L relative to the total volume of the culture medium.

9. A method of culturing NK cells, comprising the steps of: obtaining NK cells from blood isolated from an individual; and culturing NK cells in a medium for culturing NK cells as set forth in claim 1.


SEQUENCE LISTING (4 sequences)

SEQ ID NO: 1 - protein, 13 aa, Artificial Sequence
  engineered from known peptides
HGVSGHGQHGVHG

SEQ ID NO: 2 - protein, 12 aa, Artificial Sequence
  engineered from known peptides
GVSGHGQHGVHG

SEQ ID NO: 3 - protein, 8 aa, Artificial Sequence
  engineered from known peptides
SGHGQHGV

SEQ ID NO: 4 - protein, 10 aa, Artificial Sequence
  engineered from known peptides
VSGHGQHGVH